(19) United States
(12) Patent Application Publication
(10) Pub. No.: US 2025/0163490 A1
(43) Pub. Date: May 22, 2025

(54) Controlled bacteria extermination with ultra violet radiation

(71) Applicants: Noam Meir, Herzelia (IL); Itzhak Pomerantz, Kefar Sava (IL)

(72) Inventors: Noam Meir, Herzelia (IL); Itzhak Pomerantz, Kefar Sava (IL)

(21) Appl. No.: 18/956,027

(22) Filed: Nov. 22, 2024

Related U.S. Application Data
(60) Provisional application No. 63/602,093, filed on Nov. 22, 2023.

(51) Int. Cl. C12Q 1/22; C12M 1/12

(52) U.S. Cl. CPC C12Q 1/22; C12M 37/00; C12M 37/06

(57) ABSTRACT

The invention teaches a system and method for extermination of bacteria using UVC light while monitoring the quality of extermination.

### RELATED APPLICATION

This application claims priority under 35 U.S.C. § 119 (e) to U.S. Provisional Application No. 63/602,093, filed Nov. 22, 2023, which is hereby incorporated by reference in its entirety.

### BACKGROUND OF THE INVENTION

The use of UV light for extermination of bacteria, such as salmonella, from fluid food, such as tahini, is described in a provisional patent application by Meir et al No. 63/545,370 filed on 24 Oct. 2023, enclosed here as attachment A.

The system in that application does not include means for verifying that the level of bacteria in the fluid is actually reduced by the process.

Such verification is very important in in-line production processes such as food manufacturing.

Unfortunately, such in-line verification does not exist today, and verification requires taking samples to a lab for off-line testing. Such testing is time consuming, expensive, and requires expensive storage of deliverable products in expectation for quality assurance.

Also, detection of bacteria in deliverable products implies throwing away and wasting large amounts of products.

It would be very desirable to have in-line reliable means to verify the extermination of bacteria in fluid material.

### Definition of Terms

Fluidic material—Material that can be poured by gravitation, such as powder, liquid, dough, mixture of solid particles and fluid, slurry etc.
UV—Radiation of light at spectrum in the region of 100-420 nm
UVC UV radiation spectrum in the region of 200-280 nm which is known as damaging the DNA and RNA molecules of biologic pathogens such as bacteria and viruses

### SUMMARY OF THE INVENTION

The present invention makes use of the empirical fact that live bacteria generate fluorescent light at known wavelength, while dead bacteria does not generate such light.

Residual fluorescent is also created by certain proteins, as described in “Ultraviolet fluorescence detection and identification of protein, DNA, and bacteria” by P. J. Hargis, Jr., T. J. Sobering, G. C. Tisone, and J. S. Wagner et. Al.

Therefor the detection of fluorescence cannot be used as an indication of presence of bacteria. However, while UVC energy does kill bacteria in the fluid (as described in the above mentioned application), while it does not change the fluorescence of other proteins, a negative gradient in the fluorescence while exposing a fluid to UVC radiation indicates an on-going reduction in the population of bacteria, thus indicate a productive process of extermination.

This theoretical hypothesis has been demonstrated in lab tests as described in the report in attachment B.

The detection process in the monitoring station is made using a UVC light source that illuminates a segment of the tested material, detecting the fluorescent light spectrum emitted from the segment using a photometer sensor, and analyzing the gradient of the fluorescent reflection over time in a processor.

A reduction in the fluorescent reflection indicates that there are bacteria that were affected by the UVC light.

The reliability of the process is enhanced if the very same segment of the fluid is monitored for a gradient, and this can be achieved by detouring small segments of the fluid for inspection as described below.

In a preferred embodiment of the present invention, a fluid is passing a monitoring station that measures the fluorescence, then is passing under strong UVC light for sufficient extermination of bacteria as described in Attachment A, and then is passing under a second monitoring station that measures the fluorescence again.

The apparent reduction of fluorescence between the pre-extermination monitoring station and the post-extermination monitoring station is a quantitative indication of the effective extermination.

Lack of significant difference between the fluorescence in the two monitoring stations indicate that either there was no bacteria in the fluid, or that the extermination failed to reduce the bacteria level.

As there is a major operational difference between the implication of these two indications, a preferred embodiment of the invention distinguishes between them by splitting the fluid into two streams. Almost all of the fluid (“production channel”) undergoes extermination at a nominal working parameters of radiation intensity and exposure time. A very small fracture of the material—typically 1% % (“test channel”) undergoes an extreme extermination process and exposed to much stronger radiation for a much-extended exposure time. Both streams are then monitored for fluorescence. If there is no change in fluorescence in the test channel, the conclusion is that there is no bacteria present, and the production channel continues the production process. If there is a reduction in fluorescence in the test channel and also in the production channel, the conclusion is that the extermination is effective. If there is a reduction in the test channel, but not in the production channel, the conclusion is that the extermination failed.

### LIST OF DRAWINGS

FIG. 1 shows a simplified controlled extermination system.

FIG. 2 shows a split channel extermination system

FIG. 3 shows a table of analysis of results.

FIGS. 4A 4B and 4C show fluorescence signals.

FIG. 5 shows a circular monitoring embodiment

FIG. 6 shows a linear monitoring setup

### DETAILED DESCRIPTION OF THE DRAWINGS

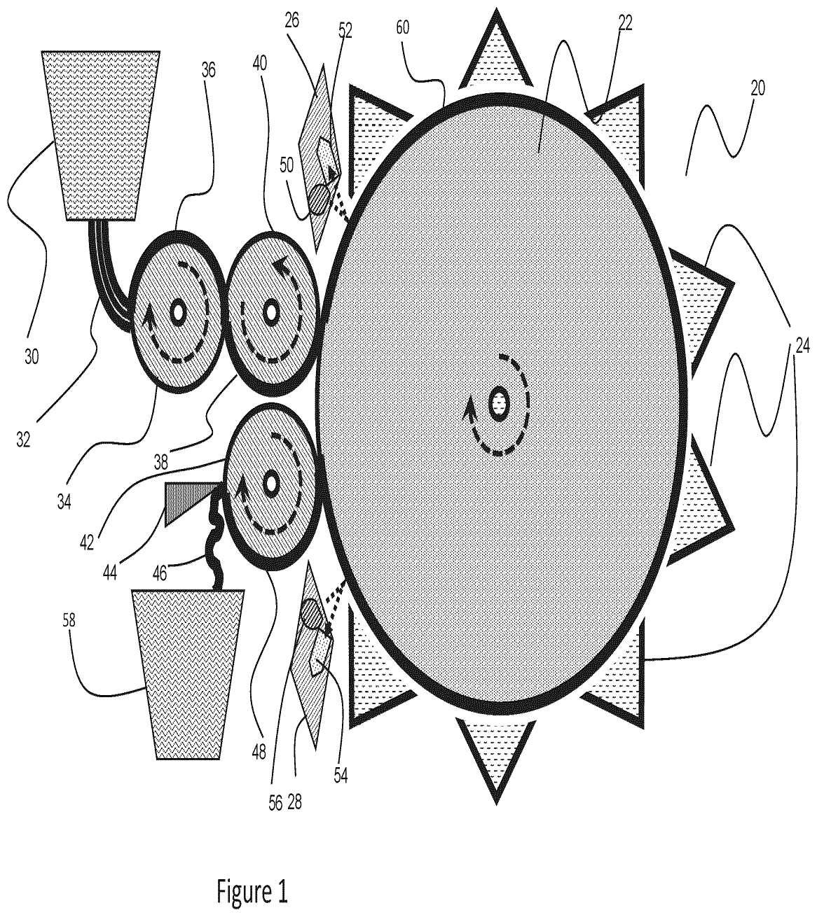

Attention is called to FIG. 1.

An extermination system 20 comprises an input container 30, an output container 58 and four tangent rotating cylinders 34, 40, 22 and 42. Such as in the 3-cylinder machine disclosed in attachment A.

Fluid material flows from the input container 30 through a feeding tube 32 and is spread over first cylinder 34 covering it with a thin layer 36 of fluid. At the tangential line between cylinder 34 and cylinder 40, most of the fluid is transferred onto cylinder 40, as a very thin layer 38. At the tangential line between cylinder 40 and cylinder 60 a very thin layer 60 of the fluid is transferred to cylinder 22.

A first monitoring station 26 radiates the fluid with UVC light 50 and monitors the fluorescent emission from the fluid 52 with a sensor as described in Attachment B. the level of fluorescence is monitored. The fluid then passes under multiple strong UVC light sources 24 as described in Appendix A.

Following the UVC radiation sources 24, the fluid passes under a second fluorescence monitoring station 28 where UVC light source 54 irradiates the fluid and sensor 56 measures the level of fluorescence emission. The fluid 48 is then transferred to cylinder 42 and is scraped by doctor blade 44 into the output container.

The level of fluorescence in the stations 26 and 28 is compared, and the reduction in fluorescence level indicates the effect of the sterilization.

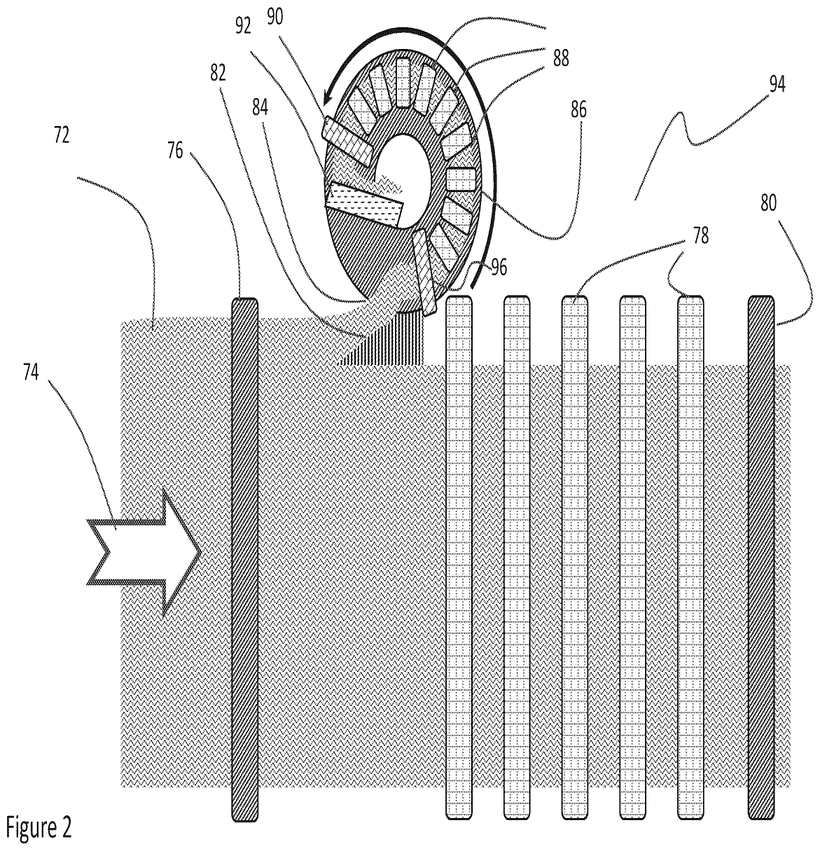

Attention is now called to FIG. 2 showing a controlled system 94.

A stream 74 of fluid 72 is split into two sub flows. Significant majority of the fluid goes under a first monitoring station 76, a series of UVC lamps 78 and a second monitoring station 80 as described in FIG. 1. A very small portion of the fluid stream is split by a blade 82 and is fed to a second extermination system having a much smaller capacity and a much more aggressive extermination. Multiple UVC sources 88 located above a wheel 86 expose the fluid to a very strong UVC radiation, much stronger than is required for extermination. The level of fluorescence is measured at a first monitoring station 96, before the exposure to UC, and the again at a second monitoring station 90 after the UVC exposure. Then the fluid is removed from the wheel by a blade 82. As the amount of test fluid is much smaller than the amount of the production fluid by several orders of magnitude, the two flows keep pace with each other.

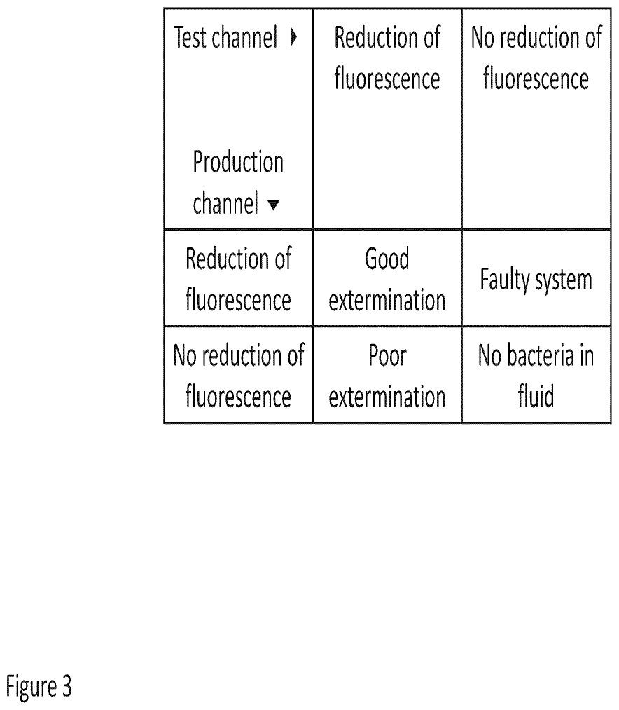

The level of fluorescence at monitoring station 90 is compared to the level of the same segment of fluid at monitoring station 96, by using the readings from the monitors with a time delay that is equal to the time it takes the fluid to travel between the two monitoring stations. This ensures that the stations 96 and 90 look at the same segment of fluid. The quality of extermination is determined according to the table of FIG. 3.

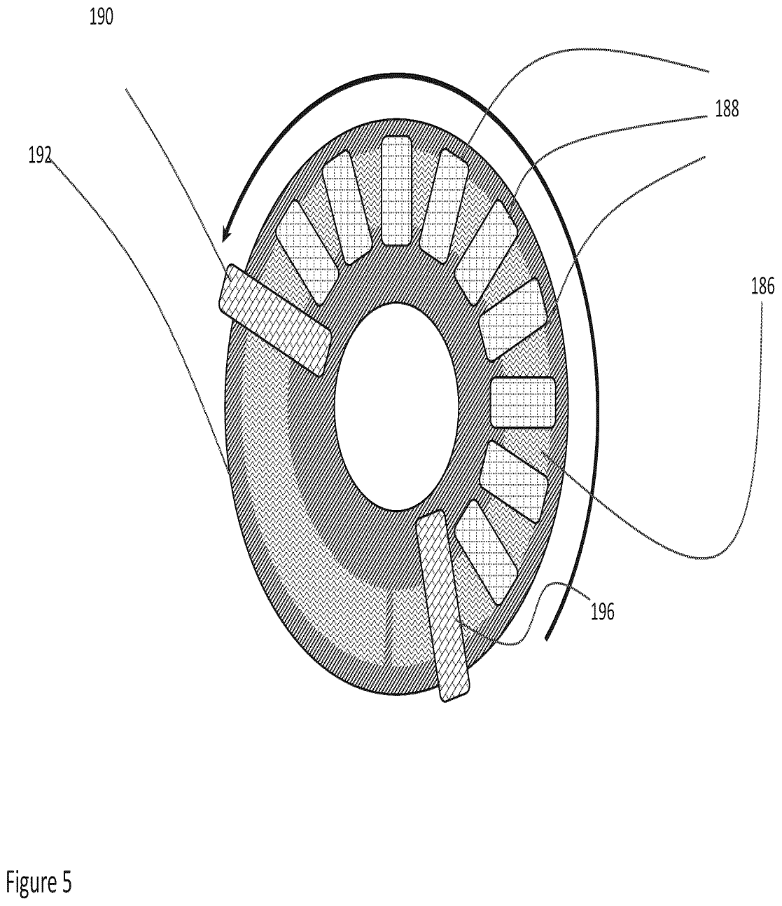

Attention is now called to FIG. 5, showing a bacteria monitoring station. An amount of fluid 186 is spread around a rotating disk 192. Two optical sensors 190 and 196, configured to detect and measure fluorescence from the fluid in response to UVC irradiation are mounted at two points above the disk. The time between the passage at the two points is known and will be called T. Several strong UVC irradiation sources 188 are mounted above the fluid. The disk is rotated and the amplitude of the fluorescence is continuously measured.

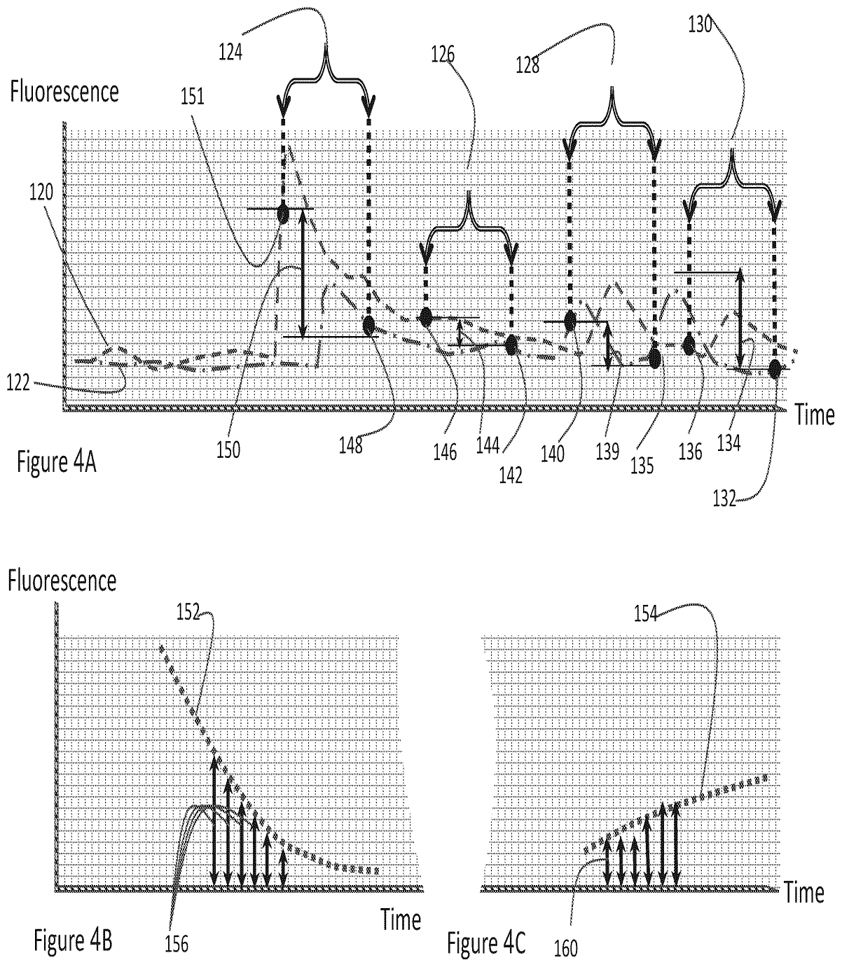

Attention is now called to FIGS. 4A and 4B showing the algorithm of monitoring the fluorescence of the reflected light.

The measured strength of the fluorescence at the first point 190 and the measured strength of the fluorescence at the second point 192 are shown on time chart.

The delay T between the two points is shown in 4 times 124, 126, 128 and 130, where the second point is the right-hand point (148, 142 etc.) and the first point is the left-hand point (151, 146 etc.) as the time between measurements 151 and 148 is exactly T, the two measurements are applied to exactly the same area of the fluid, so the difference 150 between them indicates the reduction in fluorescence due to one round trip under the irradiation setup. The other three sets of measurements are done by the same two monitoring points at different times, so that differences 144, 139 and 134 represent the reduction of the fluorescence at a certain area of the fluid as a result of the radiation. The absolute value of bacteria presence in the fluid does not need to be even.

Attention is now called to FIG. 4B. The measured values 150, 144 etc. are shown 156 on a time chart. The time difference between these measurements is constant and represents the time between samples and is typically equal to one revolution of the disk. The samples represent the fluorescence differences of a given point on the disk between cycles of the disk. Each consecutive sample is irradiated by one more pass under the irradiation area. The points 156 represents the reduction in fluorescence after a given dose of radiation. As is shown, the points can be approximated by the logarithmic pattern 152.

FIG. 4C shows the same situation as FIG. 4B, where no bacteria is present. The measured fluorescence values 160 that approximate a pattern 154 caused by proteins does not decrease by a logarithmic pattern, and actually does not decrease at all. This indicates that there are not live bacteria in the fluid.

The difference in fluorescence between time

In a preferred embodiment, the time pattern of the fluorescence level change over time is continuously cross-correlated with a decaying logarithmic pattern. If the decay in fluorescence is a result of a reduction in the UVC radiation, or reduction in the optical sensor sensitivity, or a result of dirt accumulated in the optical path-then the changes in fluorescence will not fit a logarithmic pattern. If the reduction is a result of a decaying bacteria population, then the reduction will fit a logarithmic pattern due to the natural statistics of death of populations.

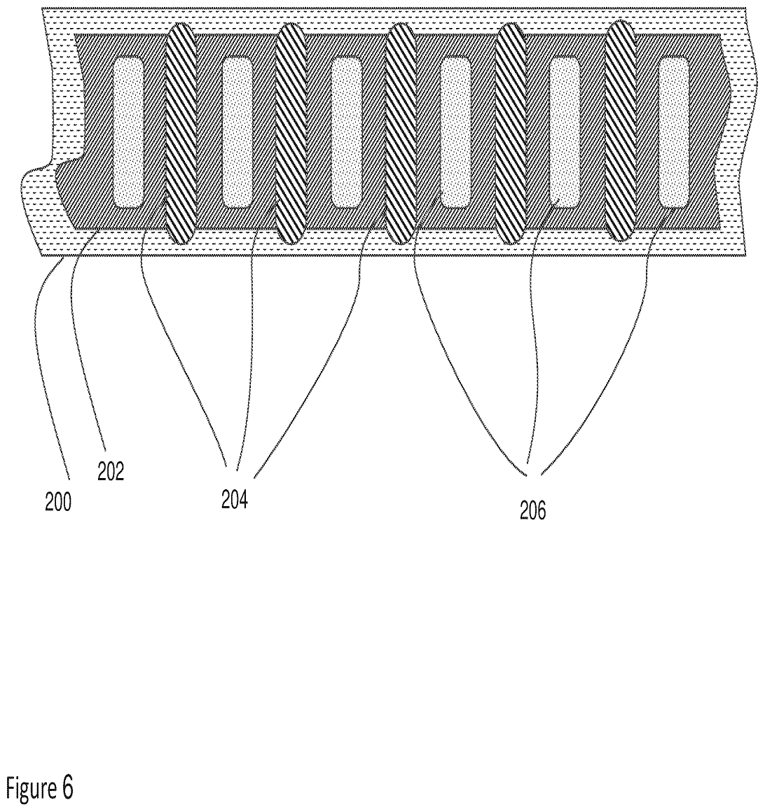

Attention is now called to FIG. 6, showing a linear monitoring system. Fluid 202 is spread on a linear conveyer 200 and is passing under a set of UVC irradiation units 204 and under deinterleaved set of fluorescence sensors 206.

The sensors are continuously sampled. A processor calculates the difference between two consecutive sensors at a delay equal to the time of travelling between the sensors-providing an indication of the change in fluorescence due to passage under the irradiation sources 204. The sequence of calculated values is examined for a logarithmic pattern. The detection of a logarithmic pattern indicates the presence of live bacteria being exterminated.

1. A system for extermination of bacteria in fluid media, comprising
a At least one UV source
b At least one fluorescence detection sensor
c means that monitor the reduction in fluorescence resulting from exposure of the fluid to UV light.

2. A system as in claim 1 wherein the fluid moves from a first fluorescence detection sensor through a light exposure area to a second fluorescence detection sensor

3. A system as in claim 1, comprising means to split the fluid into a monitoring stream and a production stream, where the test stream has a significantly lower capacity and a significantly higher light exposure intensity than the production stream.

4. A system as in claim 3 comprising two monitoring stations at two points along the fluid flow, wherein the stations are sampled at a delay that is essentially equal to the time it takes the fluid to travel between the two stations.

5. A system as in claim 3, wherein the monitoring module comprises a circular disk.

6. A system as in claim 3, wherein the monitoring module is a linear conveyer comprising deinterleaved irradiation sources and optical sensors.

7. A method of exterminating bacteria in a fluid, comprising the steps of—
a Thinning the fluid to a layer of less than 200 microns
b Measuring the fluorescence of the fluid under UVC light
c Exposing the fluid to strong UVC light
d Measuring the fluorescence of the fluid under UVC light after exposure.
e Comparing the levels of fluorescence before and after exposure and
f Evaluating the extermination based on the comparison.

8. A method of monitoring the presence of bacteria in a fluid, comprising the steps of repetitive passing under irradiation sources and optical sensors and measuring the decrease in fluorescence reflection from the fluid due to the repetitive irradiation.

9. A method as in claim 8, wherein the decrease in fluorescence is compared to a logarithmic pattern.
